(19) United States
(12) Patent Application Publication
(10) Pub. No.: US 2025/0312160 A1
(43) Pub. Date: Oct. 9, 2025

(54) Apparatus and Methods for Small Joint and Bony Defect Replacement

(71) Applicant: Paul E. Kraemer, Westfield, IN (US)

(72) Inventor: Paul E. Kraemer, Westfield, IN (US)

(21) Appl. No.: 18/920,637

(22) Filed: Oct. 18, 2024

Related U.S. Application Data
(60) Provisional application No. 63/591,301, filed on Oct. 18, 2023.

(51) Int. Cl. A61F 2/30; A61F 2/28; A61F 2/42; A61L 27/04; A61L 27/06; A61L 27/10; A61L 27/30; A61L 27/36; A61L 27/54

(52) U.S. Cl. CPC A61F 2/30; A61F 2/28; A61L 27/047; A61L 27/06; A61L 27/105; A61L 27/306; A61L 27/365; A61L 27/3654; A61L 27/54; A61F 2002/30084; A61F 2002/30985; A61F 2002/4228; A61L 2430/24; A61L 2430/34

(57) ABSTRACT

Apparatus and methods for joint and bony segment replacement may utilize additive manufacturing (e.g., 3D printing) of various anatomic constructs with PEEK/zeolite/ion (PZI) material. Heavy metal ion loading options may be utilized which may provide differing properties to different surfaces of the resultant apparatus. Moreover, using the PZI material, the apparatus being implanted in joint and bony segment replacement may favorably manipulate the biologic microenvironment in which it may be implanted. Further, through use of PZI material, current large joint replacement options also may be disrupted including, but not limited to, modifications of the joint replacement itself, and the augments and supporting joint reconstruction devices used in bone loss situations. Methods for joint and bony segment replacement may provide mixing two or more types of PZI material together through an additive manufacturing process; and introducing the mixture as part of an implantable apparatus.

### CROSS-REFERENCE TO RELATED APPLICATION

The present Application is a non-provisional of, and claims priority to, U.S. Patent Application No. 63/591,301 filed Oct. 18, 2023, the disclosure of which is incorporated by reference in its entirety.

### FIELD OF THE DISCLOSURE

The present disclosure generally relates to joint and bony segment replacement, and more particularly, to joint and bony segment replacement utilizing additive manufacturing (e.g., 3D printing) of various anatomic constructs with polyether ether ketone (PEEK)/zeolite/ion (PZI) material.

### BACKGROUND

Small joint (including, but not limited to, hand, wrist, radial head, foot, and ankle) reconstructive options are currently imperfect. Bony segment replacements (including, but not limited to, distal humerus, iliosacral segments, talus, and segmental long bones) are similarly imperfect in current iterations, which may include cadaveric sections, homologous anatomy, or 3D-printed titanium. Large joint replacement (such as hip, knee, shoulder) have not yet been totally disrupted by 3D printing of titanium. Their more complex revision/reconstructive challenges due to bone loss may utilize 3D-printed titanium or homologous cadaveric bone augmentation.

### SUMMARY

Embodiments of the present disclosure may provide apparatus and methods for joint and bony segment replacement utilizing additive manufacturing (e.g., 3D printing) of various anatomic constructs with PEEK/zeolite/ion (PZI) material. In embodiments of the present disclosure, heavy metal ion loading options may be utilized, which may provide differing properties to different surfaces of the resultant apparatus. Moreover, using the PZI material, the apparatus being implanted in joint and bony segment replacement may favorably manipulate the biologic microenvironment in which it may be implanted. Further, through use of PZI material, current large joint replacement options also may be disrupted including, but not limited to, modifications of the joint replacement itself, and the augments and supporting joint reconstruction devices used in bone loss situations.

Embodiments of the present disclosure may provide a method for joint and bony segment replacement including: utilizing additive manufacturing of one or more anatomic constructs with at least one PEEK/zeolite/ion (PZI) material. Different heavy metal ions may be used in the at least one PZI material to provide different properties to different surfaces of an implantable apparatus used in joint and bony segment replacement. The implantable apparatus may favorably manipulate a biologic microenvironment in which it is implanted. The additive manufacturing may be 3D printing. The PEEK/zeolite in the at least one PZI material may be a composite polymer derived from a hybrid of PEEK and negatively (−) charged ceramic zeolite molecules. The negatively (−) charged ceramic zeolite molecules may be negatively (−) charged ceramic aluminum silicate molecules. The ion in the at least one PZI material may include one or more of copper, zinc, silver, strontium, and sodium. The ion in the at least one PZI material may be sodium, and an ion exchange may result with a surrounding environment to provide uniform properties at all surfaces where the at least one PZI material is introduced. The ion in the at least one PZI material may be copper which encourages soft tissue formation when at the at least one PZI material is applied to cartilaginous surfaces. The ion in the at least one PZI material may be zinc, strontium, or a combination of the same which encourages bony tissue formation. The ion in the at least one PZI material may be silver which provides an antimicrobial environment.

Other embodiments of the present disclosure may provide a method for joint and bony segment replacement including: mixing two or more types of a PEEK/zeolite/ion (PZI) material together through an additive manufacturing process; and introducing the mixture as part of an implantable apparatus, wherein loaded ion functions of the PZI material may provide differing properties to a local environment in which the implantable apparatus is introduced. The method may also include pairing the two more types of the PZI material with an underlying titanium or other metallic superstructure to provide greater strength to the implantable apparatus. The ion in the two or more types of the PZI material may include one or more of copper, zinc, silver, strontium, and sodium. The two or more types of the PZI material may employ two or more different types of zeolite to provide differing surface environments. The two or more types of the PZI material may at least partially cover an underlying metallic structure. The implantable apparatus may be selected from the group consisting of: artificial joints, bone defects, partial articular surfaces, segmental defects, craniofacial reconstructions, or other mechanisms to address orthopedic, craniofacial, or skeletal reconstructive challenges. The two or more types of the PZI material may include copper droplets at articular surfaces and zinc at bony surfaces in a finger proximal interphalangeal (PIP) joint replacement. The two or more types of the PZI material may include copper at an articular surface, zine at bony attachment sites, and scattered nodes of zinc. The two or more types of the PZI material may include copper, zinc, and scattered silver in a large joint application.

### DETAILED DESCRIPTION

Embodiments of the present disclosure may provide apparatus and methods for joint and bony segment replacement utilizing additive manufacturing (e.g., 3D printing) of various anatomic constructs with PEEK/zeolite/ion (PZI) material. One example of PEEK/zeolite material is ZFUZE™ composite polymer (DiFUSION Technologies, Inc.; Austin, TX), which is a composite polymer derived from PEEK and negatively (−) charged ceramic aluminum silicate molecules. It should be appreciated, however, that other forms of negatively charged zeolites may be compounded with the PEEK polymer without departing from the present disclosure. PEEK alone is hydrophobic, which may promote an inflammatory, M1 type response. Through addition of a negatively charged zeolite, however, the composite is rendered hydrophilic and less likely to cause an inflammatory response. A balancing positively charged ion may be included. If the positively charged ion is sodium, as in ZFUZE™, the result is ion exchange with the surrounding environment, which may provide uniform properties at all surfaces of ZFUZE™. It should be appreciated, however, that there may be some surfaces of an additively manufactured apparatus in embodiments of the present disclosure that do not have uniform properties.

Various positively charged ions and combinations of the same may be used in embodiments of the present disclosure. For example, positively charged ions, including, but not limited to, copper, may encourage soft tissue formation and may be helpful for cartilaginous surfaces. Other positively charged ions including, but not limited to, zinc and strontium may encourage bony tissue formation. Silver is an example of a positively charged ion that may provide an antimicrobial environment. Regardless what positively charged ion is used, when combined with PEEK/zeolite, the resultant material may be referred to herein as PZI.

Embodiments of the present disclosure may provide a mixture of two or more types of PZI together through the additive manufacturing process. The loaded ion functions of the PZI may provide differing properties to the local environment. When greater strength may be desired, the two or more types of PZI may be paired with an underlying titanium or other metallic superstructure. The zeolite coating may bring more strength to the biologic surface; however, the majority of the strength of the apparatus may come from the underlying metallic superstructure. Accordingly, PZI may be added to extant devices including, but not limited to, surface treatments, augments, or other applications to bring biologic activity via the PZI to extant structures such as joint replacements.

It should be appreciated that multiple positively charged ions may be used in embodiments of the present disclosure. Multiple positively charged ions may include, but are not limited to, copper, zinc, silver, strontium, sodium, and combinations of the same. There also may be some embodiments where two or more different types of zeolites may be employed so as to provide differing surface environments. There also may be embodiments of the present disclosure where a single ion may be used. Further embodiments of the present disclosure may include an underlying metallic structure which may be partially or fully covered by the PZI material.

An apparatus and methods for a mixture of two or more types of PZI together through the additive manufacturing process according to embodiments of the present disclosure may be fashioned into artificial joints, bone defects, partial articular surfaces, segmental defects, or other mechanisms to address orthopedic, craniofacial, or skeletal reconstructive challenges. This could include joints such as distal humerus, radial head, hand/finger, foot and ankle, or other small joints. This could include segmental bone defects in long bone trauma, or periarticular defects such as encountered in tibial plateau fractures. However, it should be appreciated that any bony reconstructive option, including large joint, may be included in embodiments of the present disclosure. Further, craniofacial reconstructions, including additively manufactured anatomy such as zygomatic arch, skull defects, or other prosthesis, may employ the apparatus and methods according to embodiments of the present disclosure.

In an embodiment of the present disclosure, a small joint application such as finger proximal interphalangeal (PIP) joint replacement may be performed through use of PZI including copper droplets at the articular surfaces and PZI including zinc at the bony surfaces. This application may be advantageous in post-trauma reconstructive options including, but not limited to, distal humerus or finger/toe reconstructions where bone healing is important but articular surfaces must be maintained. It also should be appreciated that additively manufactured PZI may be utilized for radial head and humerus small joint reconstructions where normal anatomy has been destroyed, such as through trauma or degenerative conditions, and needs to be supplemented or replaced.

Another embodiment of the present disclosure may provide a bony segment application in a distal humerus additively manufactured bony replacement. In such an application, the articular surface may PZI using copper, the bony attachment sites may be PZI using zinc, and scattered nodes throughout may be PZI using zinc. Accordingly, different segments of the implant may be optimized relative to precise biologic microenvironment needs.

The process of utilizing different PZI analogs to customize differing surfaces may maximize local biologic activity in embodiments of the present disclosure. In an embodiment of the present disclosure, certain surfaces in a replacement of a distal humerus for a non-reconstructible intra-articular fracture may be optimized for bone ingrowth, while other surfaces may be optimized for fibrocartilage articular surfaces. However, all surfaces may include scattered zinc for anti-infective properties.

It also should be appreciated that additively manufactured PZI material may be utilized for post-traumatic defects in embodiments of the present disclosure. This may be used with the elbow, for example, but also with segmental tibia defects, metaphyseal defects (e.g., tibial plateau), and shoulder/hip bone defects in embodiments of the present disclosure. In some embodiments, these materials may be custom printed and intra-operatively modified.

Other embodiments of the present disclosure may provide a large joint application such as a shoulder replacement involving bone loss from a prior infection. In such an application, a custom additively manufactured scapular side augment may utilize different areas of the implant with copper PZI, zinc PZI, and scattered silver PZI. It should be appreciated that plasma spraying or another method of PZI attachment may be applied in these large joint applications such as hip, knee, or shoulder replacements. This may be advantageous to provide biologic characteristics to an implant that may be traditionally or necessarily made of metallic components, such as in hip and knee arthroplasty or in larger segmental bone replacement devices such as tumor prostheses.

Regardless what additively manufactured custom implants may be generated, they can be utilized with titanium implants. They may include surface treatments, screw anchor holes, and/or roughened surfaces for maximum bony contact.

Advantages of the apparatus and methods for joint and bony segment replacement utilizing additive manufacturing of various anatomic constructs with PZI material may provide customization of implant morphology/size, intra-operative flexibility in modification of an implant, customization of surface biologic activity, providing implant modulus of elasticity closer to the native bone, radiolucent or nearly radiolucent design, and/or the ability to be applied to an underlying superstructure (e.g., plasma spraying).

Although aspects of the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method for joint and bony segment replacement comprising:
utilizing additive manufacturing of one or more anatomic constructs with at least one PEEK/zeolite/ion (PZI) material.

2. The method of claim 1, wherein different heavy metal ions are used in the at least one PZI material to provide different properties to different surfaces of an implantable apparatus used in joint and bony segment replacement.

3. The method of claim 2, wherein the implantable apparatus favorably manipulates a biologic microenvironment in which it is implanted.

4. The method of claim 1, wherein additive manufacturing is 3D printing.

5. The method of claim 1, wherein the PEEK/zeolite in the at least one PZI material is a composite polymer derived from a hybrid of PEEK and negatively (−) charged ceramic zeolite molecules.

6. The method of claim 5, wherein the negatively (−) charged ceramic zeolite molecules are negatively (−) charged ceramic aluminum silicate molecules.

7. The method of claim 1, wherein the ion in the at least one PZI material comprises one or more of copper, zinc, silver, strontium, and sodium.

8. The method of claim 1, wherein the ion in the at least one PZI material is sodium, and wherein an ion exchange results with a surrounding environment to provide uniform properties at all surfaces where the at least one PZI material is introduced.

9. The method of claim 1, wherein the ion in the at least one PZI material is copper which encourages soft tissue formation when at the at least one PZI material is applied to cartilaginous surfaces.

10. The method of claim 1, wherein the ion in the at least one PZI material is zinc, strontium, or a combination of the same which encourages bony tissue formation.

11. The method of claim 1, wherein the ion in the at least one PZI material is silver which provides an antimicrobial environment.

12. A method for joint and bony segment replacement comprising:
mixing two or more types of a PEEK/zeolite/ion (PZI) material together through an additive manufacturing process; and
introducing the mixture as part of an implantable apparatus, wherein loaded ion functions of the PZI material provide differing properties to a local environment in which the implantable apparatus is introduced.

13. The method of claim 12 further comprising:
pairing the two more types of the PZI material with an underlying titanium or other metallic superstructure to provide greater strength to the implantable apparatus.

14. The method of claim 12, wherein the ion in the two or more types of the PZI material comprises one or more of copper, zinc, silver, strontium, or sodium.

15. The method of claim 12, wherein the two or more types of the PZI material employs two or more different types of zeolite to provide differing surface environments.

16. The method of claim 12, wherein the two or more types of the PZI material at least partially cover an underlying metallic structure.

17. The method of claim 12, wherein the implantable apparatus is selected from the group consisting of:
artificial joints, bone defects, partial articular surfaces, segmental defects, craniofacial reconstructions, or other mechanisms to address orthopedic, craniofacial, or skeletal reconstructive challenges.

18. The method of claim 12, wherein the two or more types of the PZI material include copper droplets at articular surfaces and zinc at bony surfaces in a finger proximal interphalangeal (PIP) joint replacement.

19. The method of claim 12, wherein the two or more types of the PZI material include copper at an articular surface, zine at bony attachment sites, and scattered nodes of zinc.

20. The method of claim 12, wherein the two or more types of the PZI material include copper, zinc, and scattered silver in a large joint application.
